(19) United States
(12) Patent Application Publication
(10) Pub. No.: US 2022/0405664 A1
(43) Pub. Date: Dec. 22, 2022

(54) SYSTEMS AND METHODS FOR MANAGING THE PERFORMANCE OF SERVICES

(71) Applicant: Physicians Software Solutions, LLC, Chicago, IL (US)

(72) Inventors: Cameron Full, Chicago, IL (US); Eric Ellis, Ely, IA (US); Jason Stoner, Dublin, OH (US); Jason Jones, Swisher, IA (US); Dale Rumer, Walford, IA (US); Jason Souyais, Fort Gratiot, MI (US); Michael Sedaghatfar, Tiburon, CA (US); Michael Pikos, Palm Harbor, FL (US)

(73) Assignee: Physicians Software Solutions, LLC, Chicago, IL (US)

(21) Appl. No.: 17/354,803

(22) Filed: Jun. 22, 2021

(51) Int. Cl. G06Q 10/06; G06Q 30/06; G06Q 10/02; G06Q 30/02

(52) U.S. Cl. CPC G06Q 10/06312; G06Q 30/0631; G06Q 10/02; G06Q 10/0639; G06Q 30/0214; G16H 40/20

(57) ABSTRACT

A system for managing the performance of services. A schedule includes slots for performing services. A referral profile database contains historical data for referral sources of the services. An incoming request module receives on an inquiry date an inquiry from a client to reserve one of the slots in the schedule for performing a requested service, and also receives one of the referral sources referring the client. A scheduling module reserves a scheduled slot among the slots for performing the requested service for the client. An analysis module determines whether the requested service has been performed and, when performed, stores a completion date corresponding thereto. The analysis module calculates a delay between the inquiry date and the completion date. An action module updates the historical data for the one of the referral sources based upon whether the requested services were performed, and based on the delay calculated.

### FIELD

The present disclosure generally relates to systems and methods for managing the performance of services, and more particularly to systems and methods for managing the performance of referred services.

### BACKGROUND

In many industries, referrals are an abundant (or even the primary) source of incoming business. These referrals may arise simply as a recommendation from a trusted professional, or in certain cases be required for insurance approval or other reasons. For example, incoming patients needing services from a periodontist or orthodontist often come by way of referral from a dentist. Generally, referrals are provided by a more generalized practitioner to a specialist, though referrals may also be made between related service providers as well. For example, a pediatrician may refer patients to a dentist, or a real estate agent to various contractors or loan officers.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

One embodiment of the present disclosure generally relates to a system for managing the performance of services. The system includes a processing system and a memory system with a schedule stored in the memory system, the schedule including slots for performing services. A referral profile database is stored in the memory system, the referral profile database containing historical data for referral sources of the services. An incoming request module is executable by the processing system and configured to receive on an inquiry date an inquiry from a client to reserve one of the slots in the schedule for performing a requested service among the services, and also configured to receive one of the referral sources referring the client. A scheduling module is executable by the processing system and configured to reserve a scheduled slot among the slots for performing the requested service for the client. An analysis module is executable by the processing system and configured to determine whether the requested service has been performed and, when the requested service is determined to be performed, store a completion date corresponding thereto. The analysis module is further configured to calculate a delay between the inquiry date and the completion date. An action module is executable by the processing system and configured to update the historical data for the one of the referral sources based upon whether the requested services were performed, and based on the delay calculated.

Another embodiment generally relates to a method for managing the performance of services. The method includes storing a schedule of slots for performing services and storing ratings for referral sources. The method further includes receiving an inquiry from a client to reserve one of the slots for performing a requested service among the services, also receiving one of the referral sources referring the client. The method further includes storing an inquiry date corresponding to when the inquiry was received from the client and reserving a scheduled slot among the slots for performing the requested service. The method further includes determining whether the requested service has been performed and, when the requested service is determined to be performed, storing as a completion date, and then calculating a delay between the inquiry date and the completion date. The method further includes changing the rating of the one of the referral sources based upon whether the requested services were performed, and based on the delay calculated.

Various other features, objects and advantages of the disclosure will be made apparent from the following description taken together with the drawings.

### BRIEF DESCRIPTION OF THE DRAWINGS

The present disclosure is described with reference to the following drawings.

FIG. 1 is a schematic view an exemplary data structure for a system according to the present disclosure.

FIG. 2 depicts an exemplary schedule for performance services according to the present disclosure.

FIG. 3 is a schematic view of a system for performing services according to the present disclosure.

FIGS. 4 and 5 depict an exemplary work flow for managing the performance of services according to the present disclosure.

FIG. 6 is a schematic view of an exemplary control system within a system according to the present disclosure.

FIG. 7 depicts an exemplary user display for selecting among ranking algorithms according to the present disclosure.

FIG. 8 depicts an exemplary report summarizing the performance of services according to the present disclosure.

### DETAILED DISCLOSURE

The present inventor has recognized that referral systems presently known in the art are inadequate, failing to enable an accurate assessment of the relationship between a service provider and various referral sources. Service providers may recognize the value in building and developing a network with referral sources to generate a steady flow of business going forward. Existing techniques for managing and developing referral are essentially limited to noting the volume of referrals received from a given referral source, whereby it is assumed that more is better.

However, the present inventors recognized that the quality of referrals received from referral sources varies greatly. For example, one referral source may provide very little education or background information to a client or customer with respect to the services they are being referred to receive from the service provider. When that client later schedules a consultation with the service provider, this lack of background information (e.g., the extensiveness of the service, estimated wait time, cost, side effects, and/or recovery time) leads to a much lower incidence of referrals being converted into actual service offerings. This in turn results in a waste of time and resources by the service provider. Additionally, this results in a longer wait time for well-informed clients trying to schedule a consultation and/or time for services to be performed.

In contrast, a high-quality referral source may provide more education and/or a more accurate portrayal of the services being referred to the client. This results in only serious clients contacting the service provider, thereby preserving service provider time and resources those having a real possibility of completing the services.

Moreover, the present inventor has recognized that by using the presently disclosed systems and methods to assess the quality of a referral source, the service provider has a better opportunity for serving the clients referred therefrom. The presently disclosed systems and methods also enable the cultivation of relationships with the various referral sources, for example by identifying those that require further education and other development on the services being offered. Likewise, the presently disclosed system and methods identify which referral sources are sufficiently developed and may not require as much time and resource allocation at the present.

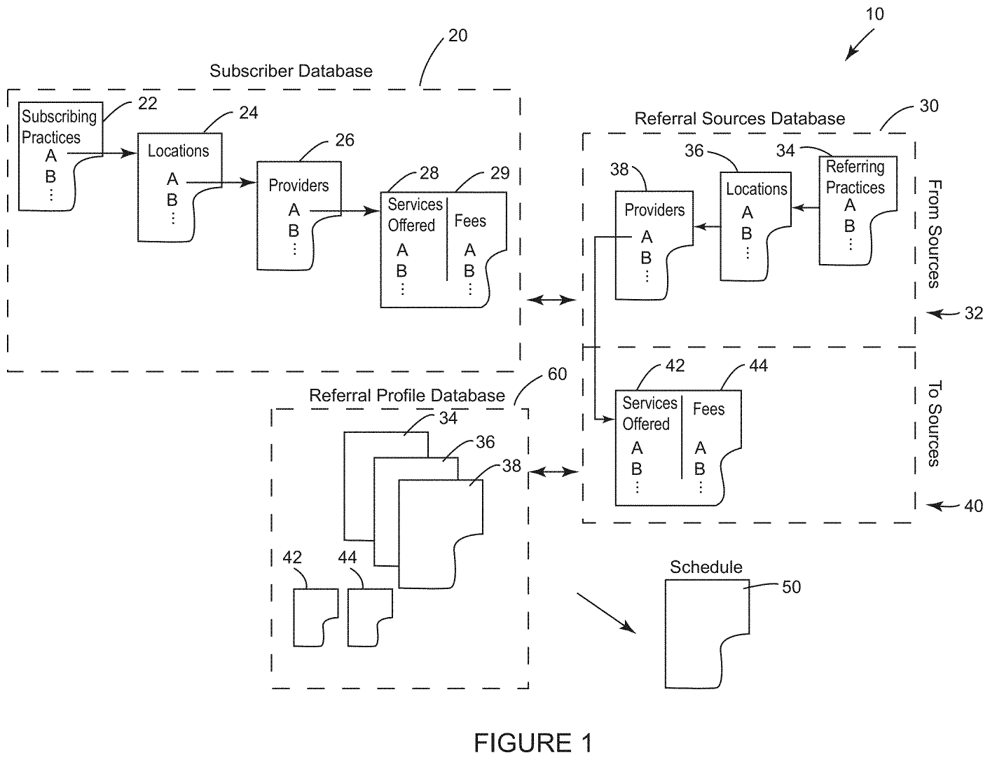

FIG. 1 depicts an exemplary data structure for a system 10 according to the present disclosure for managing the performance of services. The system 10 includes both the management of clients and referral sources. The system 10 includes a subscriber database 20 of service providers configured for using the system 10. Although these subscribers may include service providers across many industries, the present disclosure will principally provide examples relating to periodontists as the service providers for the sake of clarity and consistency throughout. In certain embodiments, the system 10 is offered to service providers as a subscription service, and thus service providers are also referred to as subscribing providers, practices, and the like.

In the subscriber database 20 shown in FIG. 1, data is stored with respect to subscribing practices 22, which may be separate subscriber businesses, such as Island Periodontic Associates or Midwest Periodontic Specialists, inc., for example. Another portion of the subscriber database 20 in this example stores locations 24 for each of these subscribing practices 22. In the example previously discussed, Island Periodontic Associates may have only a single location, whereas Midwest Periodontic Specialists may have several locations at which services are provided (e.g., across multiple cities or states). A listing of providers 26 that perform services is also stored for each of the locations 24 within the subscriber database 20. It will be recognized that certain providers may be associated with multiple locations 24. Additionally, data relating to these services offered 28 and fees 29 corresponding thereto for each of the providers 26 is stored within the subscriber database 20. By way of example, the services offered 28 data may include single or multiple implant referral examinations, general periodontal referral exam, or a gingival grafting exam.

As will become apparent, the system 10 marries and integrates information from the subscriber database 22 with that of a referral sources database 30, also shown in FIG. 1. In the exemplary referral sources database 30 shown, data may be further subdivided into from sources 32 and to sources 40, corresponding to data relating to referrals originating with the referral sources, and data corresponding to services in which the subscriber refers to the referral sources, respectively. Within the from sources 32 segment, the referral sources database 30 includes a listing of referring practices 34, for example Island Dentists or Affiliated Dentists, for example. The locations 36 in which each of the referring practices 34 is situated is also stored within the referral sources database 30, as well as a listing of the particular providers 38 associated with each of those locations 36.

In the example shown in FIG. 1, the referral sources database 30 further includes a listing of services offered 42 and fees 44 associated therewith for each of the providers 38. As will be discussed further below, this enables the system 10 to also track and manage the reverse referral of services from one of the subscribing practices 22 back to a referral source within the referral sources database 30.

FIG. 1 also shows a referral profile database 60 and schedule 50, which are described further below.

In certain embodiments, the data within the system 10 (e.g., the subscriber database 20 and/or referral sources database 30) are imported and/or managed by an internal administrator separate from both the subscribers and referrals. For example, a third-party consulting service may manage this data as part of the subscription service offered to subscribing practice. In other cases, portions of the subscriber database 20 and referral sources database 30 may be editable by subscribing service providers and/or the referral sources themselves. For example, subscribing practices and/or referral sources may have access to update the services offered 28 and fees 29 associated therewith within the subscriber database 20, and/or the services offered 42 and fees 44 associated therewith within the referral sources database 30, respectively. In other words, a particular referral source may have access and authorization to update their services offered 42 and fees 44 as they change, and as their association with providers 38, or locations 36 require updating.

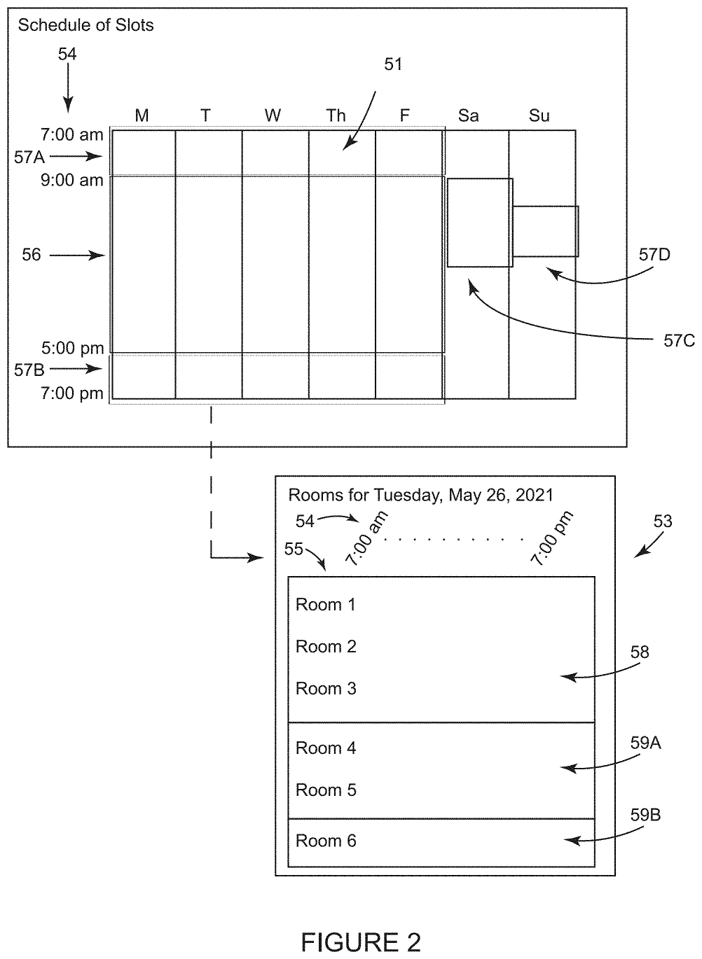

Another portion of the system 10 for managing the performance of services is maintaining and allocating slots within a schedule 50 for performing these services. FIG. 2 shows an exemplary schedule 50 with slots 51 for performing the various services offered by the service provider, shown here for a specific location of a specific subscribing practice. The schedule 50 may also be shown and managed specific to a particular provider associated with that location.

The schedule 50 includes days 52 and times 54 for each of the available slots 51 to be scheduled. In some examples, the slots 51 are further divided into standard time slots 56, which in the present example correspond to traditional hours between 9 am and 5 pm during the standard work week. Additionally, the schedule 50 shown includes preferred times 57A-57D corresponding to hours outside of the standard work day and/or week. In this manner, these preferred times 57A-57D correspond to premium offerings for the convenience of clients who cannot schedule during the standard time slot 56, for example. In certain examples, a third preferred time slot 57C and a fourth preferred time slot 57D may be configured to be even more preferred than the first and second preferred time slots 57A and 57B, as these correspond to weekend appointments. As will become apparent, configuring the schedule 50 in this manner enables the system 10 to limit the scheduling during preferred time slots 57A-57D to particular referrals when advantageous.

The schedule 50 of FIG. 2 further shows a sub menu of room listings 53 of rooms for performing services on a particular day, here Tuesday, May 26, 2021. The room listing 53 includes times 54 throughout the day, along with the various rooms 55 within the location in which the services of interest may be performed. In this manner, it will be recognized that the system 10 specifically shows these slots 51 in the schedule 50, as well as the rooms 55 and the room listing 53 that are appropriate for the service being requested. For example, if the service being scheduled requires surgery, the days 52, times 54, and rooms 55 will be restricted to only show those appropriate for surgery, rather than those appropriate for an initial consultation or follow-up that are not equipped for performing surgical services.

As with the slots 51 of the schedule 50, the rooms 55 within the room listing 53 may be further divided into standard rooms 58 and preferred rooms 59A-59B. For example, standard room slots 58 may correspond to interior rooms or those overlooking the parking lot or an adjacent building, whereas preferred rooms may be more sizeable, have better views, better amenities (e.g., a television, paraffin wax treatment equipment) and/or be more privately oriented within the location. As with the slots 51, allocation of the preferred room slots 59A-59B when scheduling enables enhanced management for performing these services, as well as management and development of referral sources and clients, as discussed further below.

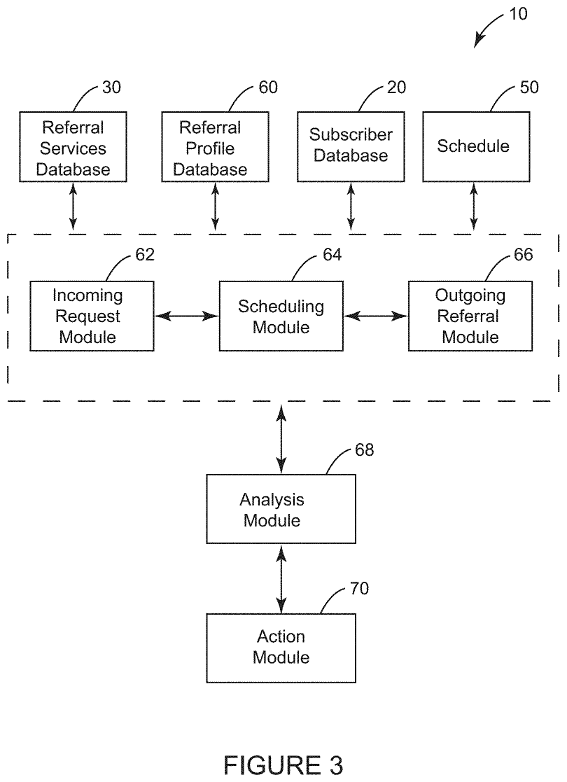

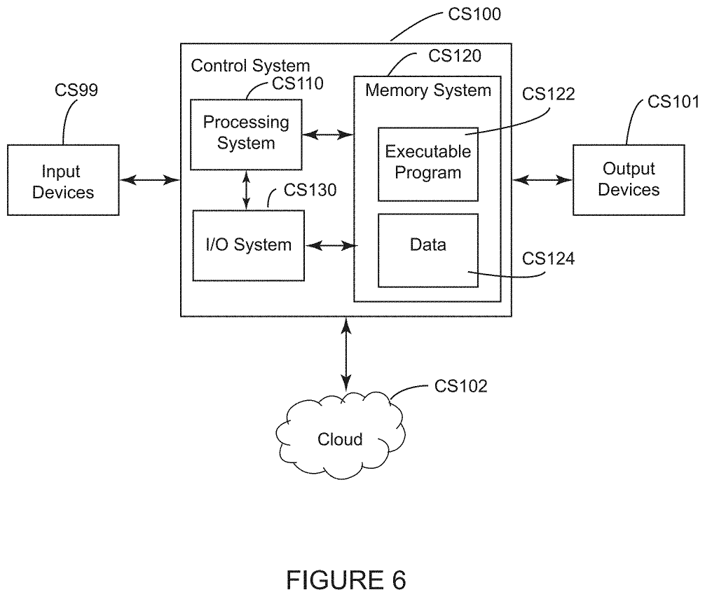

FIG. 3 depicts an exemplary information flow for a system 10 that incorporates the subscriber database 20 and the referral sources database 30 for managing the performance of services according to the present disclosure. The schedule 50, databases 20, 30, and 60, and modules 62, 64, 66, 68, and 70 are executable by the control system CS100 of FIG. 6, which is discussed further below. In particular, the schedule 50 and the databases 20, 30, 60 may be stored within the memory system CS120 of FIG. 6 to be accessible by the processing system CS110 therein.

As discussed above, the schedule 50 includes slots 51 for performing the services. In addition to the databases discussed above, the system 10 of FIG. 3 includes a referral profile database 60 also stored within the memory system CS120, which contains historical data for the referral sources stored within the referral sources database 30. In contrast to systems and methods presently known in the art, the referral profile database 60 is not merely based on the volume of referrals received by each of the referral sources. The referral profile database 60 reflects a complete history and determined quality metrics for each referral source, which is updated and grows over time.

The system 10 of FIG. 3 further includes additional modules that communicate with the databases and schedule. An incoming request module 62 is configured to receive an inquiry from a client to reserve one of the slots 51 in the schedule 50 for performing a requested service among the services offered 28 within the subscriber database 20 (FIG. 1). The date on which the inquiry from the client occurs, for example a telephone call from a prospective patient to a subscribing service provider, is also referred to as the inquiry date. The incoming request module 62 is also configured to receive information from the client relating to the referral source that referred them to the service provider. In some cases, the referral source will already be listed within the referral sources database 30, such as for a long-standing relationship or repeated business referred from a given referral source. In other cases, a new referral source will be provided by the client and added to the referral sources database 30 at the time of receiving the inquiry.

The system 10 of FIG. 3 further shows a scheduling module 64 that is executed by the processing system CS110 of the control system CS100 of FIG. 6 to execute the reserving of a schedule slot among the slots 51 for performing the requested service for that client. In certain examples, an outgoing referral module 66 is also provided within the system 10. The outgoing referral module 66 receives information relating to outgoing referrals from the service provider or subscriber to one of the referral sources. The schedule 50, databases 20, 30, 60, and modules 62, 64, 66 discussed above are further provided in communication with an analysis module 68. The analysis module 68 is configured to determine whether the requested service requested by the client has actually been performed, and when this occurred.

The analysis module 68 further determines and stores a completion date corresponding to when the requested service has actually been performed, which is used to further calculate (among other things) a delay between the inquiry date and completion date. As discussed above, one attribute of a high quality referral source is one that provides an accurate and reasonable level of background information for the service being referred, including the costs, timelines, and recovery periods of the services corresponding thereto. The present inventors have recognized that in general, higher quality referral sources tend to have a shorter average delay between completion dates and inquiry dates, as the incoming clients are better informed from the onset and are thus more serious about having the services performed. In contrast, lower quality referrals sources are more likely to refer clients that are not being particularly good candidates for the services, for example. Consequently, these clients do not end up receiving services after having a consultation. Similarly, the clients of lower quality referrals tend to take a longer period of time for consideration and/or preparation, in part because since consideration or preparation takes place after the inquiry date or consultation date) rather than before.

Under traditional systems, the referral source providing a high volume of referrals is deemed to be among the best referral sources, as they are sending a lot of potential business to the service provider. However, by tracking the elapsed times (e.g., between inquiry date, consultation date, and service completion date), conversion rates, and other metrics described herein, the system 10 presently disclosed determines which of these referral sources is high quality, and which simply send a lot of low-quality inquiries.

With continued reference to FIG. 3, an action module 70 is configured to use the outputs of the analysis module 68 to update the historical information for each referral sources. This historical information is updated based upon when these requested services are actually performed (as well as the conversion rate, the delay(s) calculated, etc.) to develop an accurate and robust referral profile database 60 based on the actual history of what has resulted from the referrals received from that referral source.

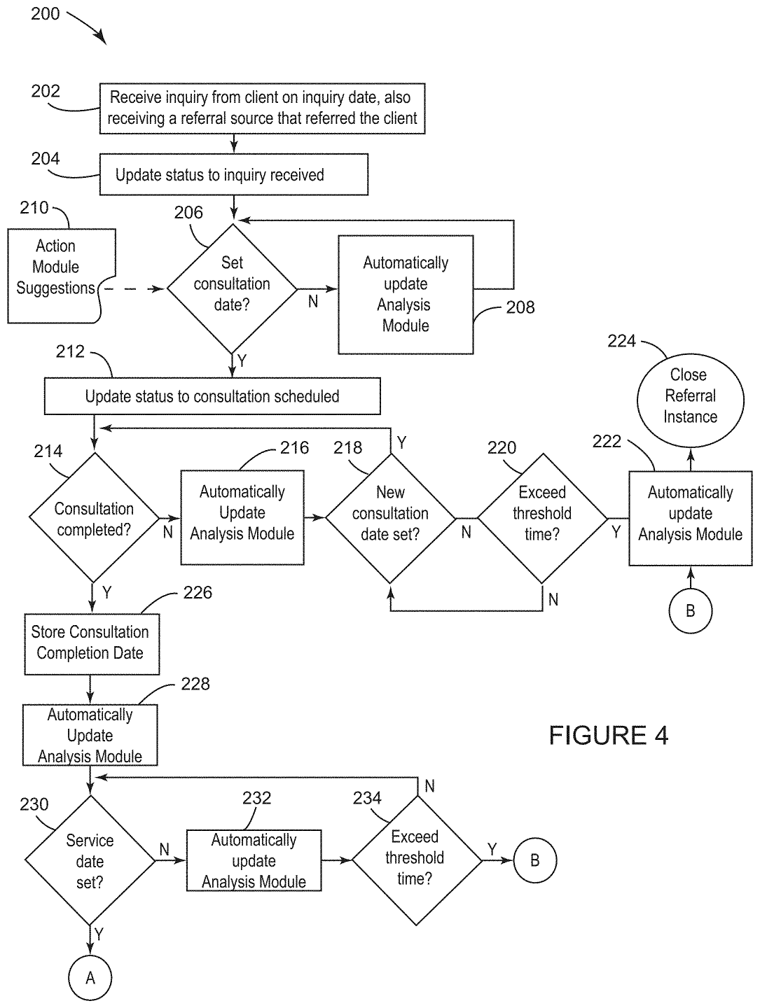

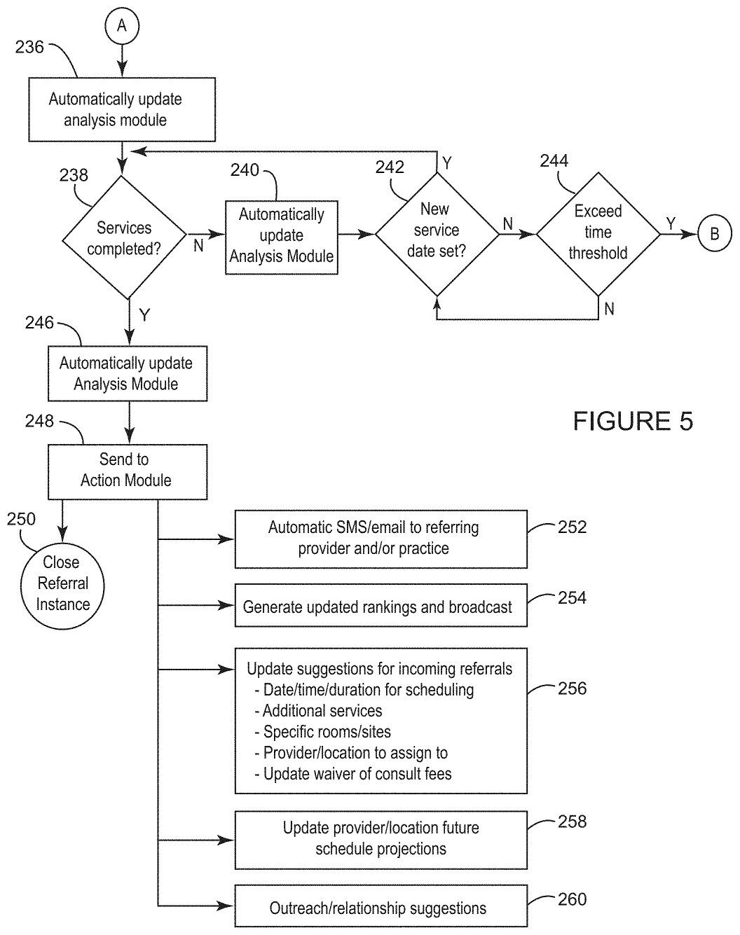

FIGS. 4 and 5 depict an exemplary process 200 for managing the performance of services according to the present disclosure, described with reference to the system 10 discussed above. In particular, the figures reflect the process between receiving an inquiry from the client and ultimately updating the referral profile database 60 in the manner previously discussed. Step 202 begins with receiving an inquiry from a client on an inquiry date (regarding a service for which they were referred by a referral source). The inquiry may be a phone call from the client to the reception desk of the service provider, for example. Information regarding the inquiry (the client, the service in question, and/or the like), as well as the inquiry date, are saved for subsequent use. The referral source referring that client is also received and stored during the inquiry. For example, a receptionist may enter all of this information into a cloud-based system for which they have access as a subscribing practice.

Step 204 provides for updating the status of the referral instance to indicate that the inquiry has been received. This may be the first status for the referral instance, or a subsequent status (e.g., if the referral source informed the subscribing practice that a referral has been made, and that the client may be inquiring). The process 200 shown here specifically provides for a situation in which a consultation is required before the services are actually performed. This may not be required by all service providers, and/or may vary by the specific service of inquiry, whether the client has seen the service provider before, or other factors. Accordingly, step 206 next provides for determining whether a consultation has been set. If not, the analysis module is automatically updated at step 208 to reflect this status and the process repeats. In certain embodiments, the action module 70 may provide suggestions in step 210 when scheduling a consultation, and/or when determining how to respond if step 206 determines that no consultation date has been scheduled. For example, the action module 70 may automatically close the referral instance (or recommend the administrator do so) after a predetermined amount of time has elapsed. Alternatively, the client may be contacted automatically as a follow up, or the system 10 may prompt an administrator to contact the client to determine whether they still wish to proceed with scheduling a consultation.

The suggestions from the action module 70 may vary based on the service provider, service of interest, or client, and/or may vary based on the historical information for the referral source stored in the referral profile database 60. For example, a repeat customer may receive automated email prompts to schedule a consultation, a referral instance may be closed after 2 months if received from a client referred by a low quality referral source (e.g., one having a conversion rate of less than 10%), and/or may recommend the administrator, professional assistant, or the practitioner herself or himself contact the client is received from a relatively new referral source (e.g., less than 5 referrals). In other examples, referring providers with a 3rd or 4th quartile overall score, lower conversion rates, lower average case values, and/or lower total referred volume may either have accelerated or decelerated follow-up (manual reminders or automated reminders to the patient, calls to patient or referral, emails to patient or referral, texts to patient or referral, even patient status letters printed & mailed to referring source for conversion reinforcement), depending upon whether the subscriber is seeking efficiency or growth trajectory. The present inventors have found that the personally touch of a live phone call can make a particularly good impression on the client. While requiring more upfront time, this in turn can be highly valuable in establishing a good relationship with the underlying referral source.

Once the consultation date has been determined to be set in step 206, step 212 provides for updating the status of the referral instance to reflect that the consultation has been scheduled. The process then continues to step 214, which determines whether the consultation has been completed. If not, the analysis module is automatically updated at step 216 to reflect this fact, which may negatively impact the quality of the referral source within the referral profile database 60. Similar historical information can also be stored for the client, which enables the service provider so ascertain who tends to cancel or altogether skip appointments.

The process continues with step 218, which determines whether a new consultation date has been set. If not, step 220 determines whether a threshold time has been exceeded. If this threshold has also not been exceeded, the process returns to step 218 to determine whether a new consultation date has been set. If instead in step 220 the threshold is determined to have been exceeded, the analysis module 68 is automatically updated to reflect this fact in step 222, and the referral instance in the present example is closed at step 224. Once again, this may reflect poorly on the referral source within the referral profile database 60. Action module 70 suggestions may also be incorporated in the process of rescheduling (including suggesting against rescheduling) and any other decision point of the process 200, based on the powerful historical information available.

Once the consultation has been determined to be completed in step 214, the process continues to step 226, whereby the consultation completion date is stored and the analysis module 68 is automatically updated to reflect this fact in step 228. The process continues to step 230, which determines whether a service date has been set for actually performing the services. If not, the analysis module 68 is automatically updated at step 230 to reflect this fact, and continues to step 234 where it is determined whether the threshold time has been exceeded. If the threshold time has not been exceeded, the process turns to step 230. In contrast, if the threshold time has been exceeded at step 234, the process proceeds to B, whereby the analysis module 68 is automatically updated at step 222 and the referral instance is closed at step 224. As discussed above, other actions may be suggested by the action module 70 rather than immediately closing the referral instance.

If instead the service date has been set as determined in step 230, the process continues on to FIG. 5, whereby step 236 provides for automatically updating the analysis module 68 to reflect this fact. The process continues to step 238, which determines whether the services have been completed. If not, the process continues to step 240, whereby the analysis module 68 is automatically updated to reflect this fact. Further, step 242 provides for determining whether a new service date has been set, if so the process returns to step 238 and if not step 244 determines whether a threshold time has been exceeded. If a threshold time has been exceeded, the process proceeds with B, which as shown on FIG. 4 results in step 222 automatically updating the analysis module 68, and then closing the referral instance in step 224. If instead in step 244 (FIG. 5) has not yet been exceeded, the process continues at step 242.

Once the services are determined to be completed at step 238, the process continues to step 246, whereby the analysis module 68 is automatically updated to reflect this fact. The process then continues to step 248, whereby the information collected and analyzed by the analysis module 68 is sent to the action module 70 in step 248. The referral instance is also closed in step 250 as these services have been completed. However, the action module 70 here performs additional steps. In the example shown, the action module 70 may automatically send a message or other indication to the referral source in step 252 (via SMS or email, for example) indicating that the services have been completed. The message or indication may be sent to the referral source at any level, including the provider, location, or referral practice as a whole. The present inventors have found that this message or indication at step 252 is particularly helpful from an electronic medical record (EMR) perspective, but also lets the referral source know that the client has followed through and performed these services. In the case of a new referral source, for example, this may prompt them to ask the client how the experience was, for example. Step 252 may further include a “thank you” message to the referral source for providing a valuable referral.

With continued reference to FIGS. 4 and 5, step 254 provides for generating updated rankings and broadcasting these rankings to one or more of the referral sources, which may be combined with the automatic messaging of step 252. For example, as the services are been completed and the referral profile database 60 is updated, the relative rankings of the referral sources may change. These rankings may be retained internally, or included (in whole or in part) with messages sent to the referral sources (e.g., “You were among the top 10 referral sources this month!” or “Congratulations, you are ranked #2 among referral sources for this service provider!”).

Step 256 provides another exemplary action by the action module 70, here by providing updated suggestions for increasing further incoming referrals. For example, a given referral source's historical information in the referral profile database 60 may result in different suggested dates, times, and even durations for scheduling consultations and/or services for subsequent clients from the same referral source (and/or repeat clients themselves). For example, if a client is a referred from a new referral source, preferred times or dates, and/or extended duration appointments, may be provided. The present inventors have configured the system and methods disclosed herein in this manner such that the service provider can make a particularly good impression on these early referrals, with the hope that this is reflected back to the referral source and thus generates good will and further return business.

The suggestions of the action module 70 at step 256 may further include generating communications to the referral source to educate them on additional services offered by the service provider, whether relevant for the present client or others. For example, for referral sources determined to be new and/or or relatively low quality (e.g., low or falling rank), informational materials may be automatically sent to try to increase the knowledge and awareness of the referral source. These materials also serve as marketing. Additional actions in step 256 may include recommending that the service provider send outbound or reverse referrals back to the referral source, for example if that referral source is determined to have a low or falling frequency of sending referrals.

In certain configurations, the step 256 may further include updating suggestions for the action module to suggest assigning future inquiries for scheduling with a particular provider or location, or to offer a waiver of consultation fees. For example, if the system 10 determines that a particular referral source is of very high quality, and generates leads with a high conversion rate, the system may recommend waiving consultation fees during the original inquiry call from the actional module suggestions 210 shown in FIG. 4, encouraging further referrals going forward. Other suggestions by the action module may include engaging particular treatment coordinators for case presentation and different patient coordinators for consulting on scheduling.

Additional actions by the action module in step 258 include updating the provider or location specific schedule projections, and also in step 260 generating suggestions for further outreach or enhancement of relationships with referral sources. In some examples, the system is configured to identify referral sources for particular continuing education courses, depending upon the types of services that are identified as not being referred by that referral source, and/or those having low conversion rates (e.g., sending marketing outreach materials to a referral source that does send implant referrals. This may also or alternatively include automatically triggered status update communications when a referral is becoming stale (has not been converted for longer than a threshold time). Likewise, communication may be prompted or automatically generated when a highly rated referral source (e.g. top 25%) does not send referral for a certain number of days (has become dormant and requires “checking in”), and/or when a new provider or practice is entered into system, an automated notification to send a “thank you” communication, for example.

FIG. 6 depicts an exemplary control system CS100 used within the system 10 and/or to perform the process 200 described above. Certain aspects are described or depicted as functional and/or logical block components or processing steps, which may be performed by any number of hardware, software, and/or firmware components configured to perform the specified functions. For example, certain embodiments employ integrated circuit components, such as memory elements, digital signal processing elements, logic elements, look-up tables, or the like, configured to carry out a variety of functions under the control of one or more processors or other control devices. The connections between functional and logical block components are merely exemplary, which may be direct or indirect, and may follow alternate pathways.

In certain examples, the control system CS100 communicates with each of the one or more components of the system 10 via a communication link CL, which can be any wired or wireless link. The control module CS100 is capable of receiving information and/or controlling one or more operational characteristics of the system 10 and its various sub-systems by sending and receiving control signals via the communication links CL. In one example, the communication link CL is a controller area network (CAN) bus; however, other types of links could be used. It will be recognized that the extent of connections and the communication links CL may in fact be one or more shared connections, or links, among some or all of the components in the system 10. Moreover, the communication link CL lines are meant only to demonstrate that the various control elements are capable of communicating with one another, and do not represent actual wiring connections between the various elements, nor do they represent the only paths of communication between the elements. Additionally, the system 10 may incorporate various types of communication devices and systems, and thus the illustrated communication links CL may in fact represent various different types of wireless and/or wired data communication systems.

The control system CS100 may be a computing system that includes a processing system CS110, memory system CS120, and input/output (I/O) system CS130 for communicating with other devices, such as input devices CS99 and output devices CS101, either of which may also or alternatively be stored in a cloud 1002. The processing system CS110 loads and executes an executable program CS122 from the memory system CS120, accesses data CS124 stored within the memory system CS120, and directs the system 10 to operate as described in further detail below.

The processing system CS110 may be implemented as a single microprocessor or other circuitry, or be distributed across multiple processing devices or sub-systems that cooperate to execute the executable program CS122 from the memory system CS120. Non-limiting examples of the processing system include general purpose central processing units, application specific processors, and logic devices.

The memory system CS120 may comprise any storage media readable by the processing system CS110 and capable of storing the executable program CS122 and/or data CS124. The memory system CS120 may be implemented as a single storage device, or be distributed across multiple storage devices or sub-systems that cooperate to store computer readable instructions, data structures, program modules, or other data. The memory system CS120 may include volatile and/or non-volatile systems, and may include removable and/or non-removable media implemented in any method or technology for storage of information. The storage media may include non-transitory and/or transitory storage media, including random access memory, read only memory, magnetic discs, optical discs, flash memory, virtual memory, and non-virtual memory, magnetic storage devices, or any other medium which can be used to store information and be accessed by an instruction execution system, for example.

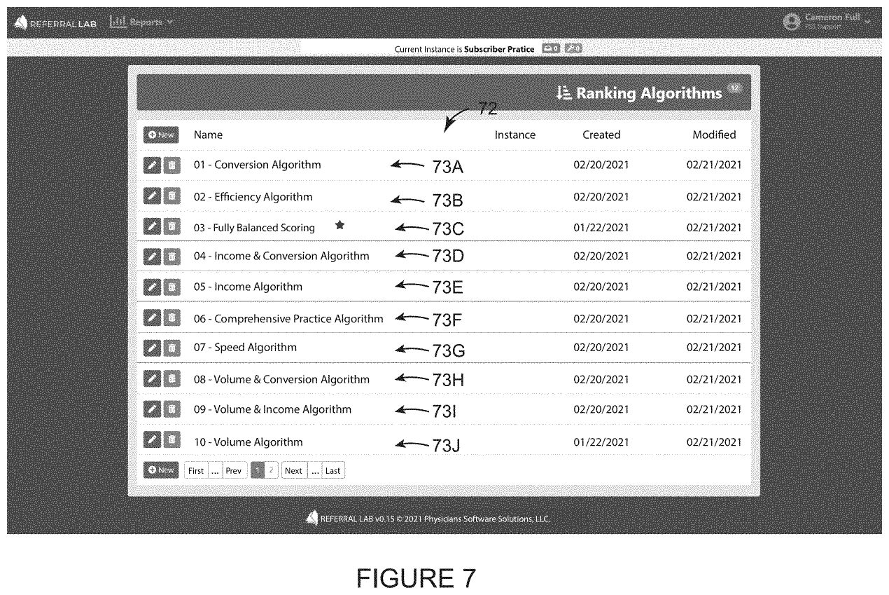

FIG. 7 depicts a screen on a graphical user interface for creating and selecting ranking algorithms for ranking the referral sources within the referral profile database 60. The example provided shows a rank configuration menu 72 including 10 algorithms 73A-73J. For example, the first algorithm 73A provides particularly heavy weighting to the conversion rate of a given referral sources between a client inquiring about services and the services actually being completed. In contrast, the seventh algorithm 73G provides particular heavy weighting for the speed between the inquiry date and actually completing the service. The rank configuration menu 72 allows the user to choose among the 10 algorithms 73A-73I, to edit them, or to create new algorithms based on what is important to the particular subscribing practice. By changing the ranking algorithm, the determination of which referral sources are high quality, average quality, and low quality varies. As discussed above, this impacts the rankings provided to the subscribing practice, but also potentially what is communicated to the referral sources, what is suggested by the action module 70 when scheduling clients referred by a referral source, and/or the like.

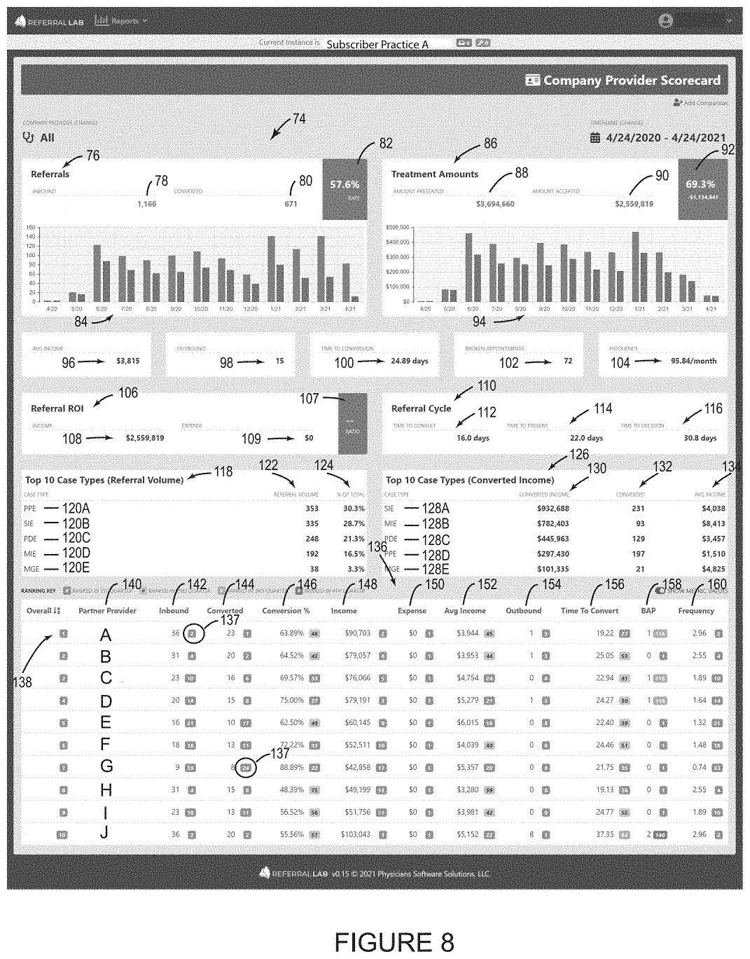

FIG. 8 depicts an exemplary referral profile database report 74 generated by the analysis module 68 and stored within the referral profile database 60. The referral profile database report 74 provides a snapshot view to the provider for the services performed via referral, as well as the quality of the referral sources in referring these services. In the example shown, a referral accounts window 76 shows a number of inbound 78 and converted 80 referrals for a given period of time, shown here as a rolling 12 month window period. A rate 82 is also provided, as well as charts of the inbound and converted numbers by time 84. Similarly, a treatment amounts window 86 is provided that shows the amount presented 88 versus the amount accepted 90 for the amount of services recommended during consultation versus those actually completed, as well as a rate 92 therebetween. A chart is again shown for the amount presented 88 and the amount accepted 90 over a time period 94.

The referral profile database report 74 further includes such metrics as an average income 96 per client, a number of outbound referrals 98 sent from the subscriber to other practices, a time for conversion 100 between the inquiry date and the completion date of performing services, a counting of broken appointments 102, and a frequency 104 of receiving referrals over a particular time period, shown here as per month.

The referrals profile database report 74 further includes a referral ROI window 106 that includes the amount of income 108 received via referral, and the amount of expense 109 spent in developing the referral sources, shown here as 0 dollars. Accordingly, the ratio 107 is presently shown as being nonexistent as there is no expense incurred in receiving the referral 108. A referral cycle window 110 is also shown, which specifically details a time to consult 112 between the inquiry date and the consultation date, a time to present 114 between. In the example shown, “time to present” is the time between referral entry date and the date the treatment presentation happens (e.g., often moderate or more involved require the provider to prepare the entirety of the treatment, as well as billing options, to present back to the patient). Likewise, “time to decision” is the amount of time between the referral entry date and the actual date recorded as to when a patient reached a terminal status point (e.g., treatment declined, treatment accepted).

The exemplary referral profile database report 74 at FIG. 8 further shows top ten cases by referral volume 118, which shows the top cases 120A-120E or types of services referred, along with the volume 122 and relative percent of referrals 124 corresponding thereto. Similarly, a top ten cases by income 126 is also shown which arranges the cases 128A-128E by income, also showing the actual converted income 130, converted number 132 and average income 134 per conversion corresponding thereto.

The referral profile database report 74 further shows a ranking of the referral sources, specifically based on the selected algorithm of the rank configuration menu 72 of FIG. 7. In this case the referral source rankings 136 include an overall rank 138, showing numbers between 1 and the number of referral sources having ranks. The referral source rankings 136 further includes a name 140 of each referral source, a number of inbound 142, and converted referrals 144, and a conversion rate 146 of referrals coming from that referral source, as well as the income 148 generated therefrom. Individual sub rankings 137 are also shown for each of the discrete values, for example with the seventh service provider G having a converted referral number 144 at ranks 24^{th} overall for the sub rating 137. The source rankings 136 further includes expenses 150 incurred for each of these referral sources, as well as an average income 152 for referrals therefrom, outbound cases 154 sent to that referral source, a time to convert 156 average for that referral source, a BAP 158 (BAP representing Broken Appointments: no shows, cancellations, or those otherwise removed from the referral cycle), in a frequency 160 of receiving referrals from that referral source.

The functional block diagrams, operational sequences, and flow diagrams provided in the Figures are representative of exemplary architectures, environments, and methodologies for performing novel aspects of the disclosure. While, for purposes of simplicity of explanation, the methodologies included herein may be in the form of a functional diagram, operational sequence, or flow diagram, and may be described as a series of acts, it is to be understood and appreciated that the methodologies are not limited by the order of acts, as some acts may, in accordance therewith, occur in a different order and/or concurrently with other acts from that shown and described herein. For example, those skilled in the art will understand and appreciate that a methodology can alternatively be represented as a series of interrelated states or events, such as in a state diagram. Moreover, not all acts illustrated in a methodology may be required for a novel implementation.

This written description uses examples to disclose the invention, including the best mode, and also to enable any person skilled in the art to make and use the invention. Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

What is claimed is:

1. A system for managing the performance of services, the system comprising:
a processing system and a memory system;
a schedule stored in the memory system, the schedule including slots for performing services;
a referral profile database stored in the memory system, the referral profile database containing historical data for referral sources of the services;
an incoming request module executable by the processing system and configured to receive on an inquiry date an inquiry from a client to reserve one of the slots in the schedule for performing a requested service among the services, and also configured to receive one of the referral sources referring the client;
a scheduling module executable by the processing system and configured to reserve a scheduled slot among the slots for performing the requested service for the client;
an analysis module executable by the processing system and configured to determine whether the requested service has been performed and, when the requested service is determined to be performed, store a completion date corresponding thereto, wherein the analysis module is further configured to calculate a delay between the inquiry date and the completion date; and
an action module executable by the processing system and configured to update the historical data for the one of the referral sources based upon whether the requested services were performed, and based on the delay calculated.

2. The system according to claim 1, wherein the historical data includes ratings for the referral sources.

3. The system according to claim 2, wherein the ratings are based in part on percentages of the requested services being performed for each of the referral sources.

4. The system according to claim 2, wherein the ratings are based in part on the delays calculated for each of the referral sources.

5. The system according to claim 2, wherein the action module is further configured to automatically communicate the ratings to at least a portion of the referral sources.

6. The system according to claim 1, wherein the action module is further configured to generate a scheduling recommendation based on the historical data of the one of the referral sources in the referral profile database, and wherein the scheduling recommendation is communicated to the scheduling module for reserving the scheduled slot.

7. The system according to claim 6, wherein the historical data includes first referral dates of the referral sources.

8. The system according to claim 6, wherein the slots in the schedule include preferred slots, and wherein the preferred slots are only reservable by the scheduling module when included in the scheduling recommendation.

9. The system according to claim 6, wherein the scheduling recommendation includes a recommended duration for reserving the scheduled slot.

10. The system according to claim 9, wherein the historical data includes first referral dates of the referral sources, wherein the action module is configured to increase the recommended duration when the first referral date of the one of the referral sources is recent.

11. The system according to claim 6, wherein the services are providable by multiple providers, and wherein the action module is further configured to include a recommended provider among the multiple providers with the scheduling recommendation.

12. The system according to claim 6, wherein the services are providable in multiple rooms, and wherein the action module is further configured to include a recommended room among the multiple rooms with the scheduling recommendation.

13. The system according to claim 6, wherein the services are providable at multiple locations, and wherein the action module is further configured to include a recommended location among the multiple locations with the scheduling recommendation.

14. The system according to claim 1, wherein the analysis module is configured to determine an elapsed time since the incoming request module received a last referral date from the one of the referral sources, and wherein the action module is further configured to automatically communicate with the one of the referral sources when the elapsed time exceeds a threshold.

15. The system according to claim 1, wherein the scheduling module is further configured to reserve a consultation slot among the slots for performing a consultation before scheduling the scheduled slot for performing the requested service, wherein the analysis module is further conFIGURE to determine a consultation delay between the consultation being performed and the requested service being performed, and wherein the action module is further configured to change the historical data of the one of the service providers based on the consultation delay.

16. The system according to claim 15, wherein the analysis module is further configured to determine a frequency in which the requested service remains unperformed after performing the consultation for the one of the referral sources, and wherein the frequency is included in the historical data of the referral profile database.

17. The system according to claim 16, wherein the action module is further configured to automatically recommend sending training materials to the one of the referral sources when the frequency corresponding thereto exceeds a threshold.

18. The system according to claim 1, wherein the scheduling module is further configured to reserve a consultation slot among the slots for performing a consultation before scheduling the scheduled slot for performing the requested service, wherein the action module is further configured to generate a consultation scheduling recommendation based on the historical data of the one of the referral sources in the referral profile database, and wherein the consultation scheduling recommendation is communicated to the scheduling module for reserving the consultation slot.

19. The system according to claim 18, wherein the historical data includes ratings for the referral sources, wherein the action module is further configured to automatically recommend a consultation fee waiver when the rating for the one of the referral sources exceeds a threshold.

20. A method for managing the performance of services, the method comprising:
storing a schedule of slots for performing services;
storing ratings for referral sources;
receiving an inquiry from a client to reserve one of the slots for performing a requested service among the services, also receiving one of the referral sources referring the client;
storing an inquiry date corresponding to when the inquiry was received from the client;
reserving a scheduled slot among the slots for performing the requested service;
determining whether the requested service has been performed and, when the requested service is determined to be performed, storing as a completion date;
calculating a delay between the inquiry date and the completion date; and
changing the rating of the one of the referral sources based upon whether the requested services were performed, and based on the delay calculated.
